(19) United States
(12) Patent Application Publication
(10) Pub. No.: US 2011/0082560 A1
(43) Pub. Date: Apr. 7, 2011

(54) PROSTHETIC BODY FOR AN UPPER LEG PROSTHETIC

(76) Inventors: Franz Copf, Kusterdingen (DE); Desirée Copf, Kusterdingen (DE); Peter Copf, Leinfelden-Echterdingen (DE)

(21) Appl. No.: 12/922,885

(22) PCT Filed: Mar. 16, 2009
(86) PCT No.: PCT/EP09/01929

§ 371 Date: Dec. 13, 2010

(30) Foreign Application Priority Data
Mar. 17, 2008 (DE) 10 2008 014 466.5

(51) Int. Cl. A61F 2/38; A61B 17/16

(52) U.S. Cl. 623/20.36; 623/20.35; 606/85

(57) ABSTRACT

The invention relates to a prosthetic body for an upper leg prosthetic comprising a shaft (5, 5a, 5b) having a proximal support plate (4) for a prosthetic head (15), wherein at least two proximally spaced and distally converging shaft posts (5a, 5b) are mounted on the support plate (4), descending below the same and connected by struts (6), particularly diagonal struts (6), and wherein cavities (12), and/or free open regions (12) are formed between the shaft posts (5a, 5b) and the struts (7), into which spongy bone can grow, wherein at least the lower connecting area of the support plate (4) whereon the shaft posts (5a, 5b) are mounted is disposed horizontally, the support plate (4), extending medially over the cross section of the shaft (5, 5a, 5b), immediately below the connection areas, and the support plate (4) transitioning ventrally and dorsally into the shaft (5, 5a, 5b) without protruding. The invention further relates to a rasp instrument for a prosthetic body and a vibration device for generating mechanical vibrations in order to induce the same in the prosthetic body.

The invention relates to a prosthetic body for an upper leg prosthesis, comprising a shaft with a proximal support plate for a prosthesis head, wherein at least two proximally spaced and distally converging shaft posts are mounted on the support plate, descending below the same, particularly connected to one another at their distal end areas, which shaft posts are particularly connected by struts, such as diagonal struts, and wherein cavities and/or freely open areas are formed between the shaft posts and the optionally provided struts, particularly into which spongy bone can grow. The profile of the shaft posts corresponds to the anatomy of the bone, and the shaft posts enlarge the surface of the prosthetic shaft with a minimal use of metal. Each of the shaft posts can have a material cavity along its extension, particularly medially or laterally, and can therefore also be understood as two connected parallel shaft posts.

The invention further relates to a rasp instrument for a prosthetic body of this type, having a surface which has rasp projections, with the shape of the surface matching that of the surface of the prosthetic body. Rasp instruments of this type are used to produce a cavity in the spongy part of the bone, which is surrounded by the cortical bone, in the proximal region of an upper leg bone (femur) following a resection, by rasping out the spongy bone to allow the prosthetic body to be positioned in the cavity.

Prosthetic bodies of the above-described type are known in the prior art and in the described embodiment form a prosthetic body which grows into the bones, i.e., in which the spongy bone completely fills the cavities and/or free open regions between the shaft posts and the struts after a healing period, so that a prosthetic body of this type is securely anchored in the bone, without having to be cemented in place.

The directional information used in this patent specification refers to the position of the prosthetic body after it has been inserted into the femur of a human who is standing upright.

In a prosthetic body as described here, it can also be provided that the shaft posts are equipped with anchoring bands, particularly annular anchoring bands, spaced along their longitudinal extension. Such anchoring bands can be embodied as radially thicker areas, which serve to enlarge the surface, which can also be achieved through other geometric shapes. For example, the anchoring bands can also be embodied as grooves. In addition to the shaft posts, the optionally provided struts can also have anchoring bands of this type.

Prosthetic bodies of this type are ordinarily made of a titanium alloy or of chromium/cobalt/molybdenum alloys, or other suitable materials, and can also have a microporous, roughened surface, which can be generated by blasting with glass beads/particles, or by etching, for example, to enable a better connection with the bone mass.

With the described construction, in which the shaft posts are spaced in the upper area where they are connected to the support plate, and converge with one another toward the bottom, and are particularly connected to one another at the distal end, wherein the posts can have a curved profile, a shaping is achieved which corresponds to the inner, open area of the proximal end of a femur, after the spongy bone structure has been rasped out. In this manner, a prosthesis of this type is optimally adapted to match the interior of the bone.

In the known prior art for this type of prosthesis, the support plate extends on all sides beyond the shaft, or the cross-section of the shaft, which is located directly below the area of connection between support plate and shaft posts. For instance, one known support plate of previous prosthetic bodies has a collar protruding on all sides in relation to the shaft of such a prosthetic body. As has long been recognized, the advantage of a collar of this type is that it can be supported on all sides against the cortical bone of the femoral shaft.

It has since been discovered that, due to inadequate knowledge regarding the physiological properties of the transference of force in the femur, the support plate of the prosthetic body heretofore known in the art has been designed as too large. Frequently, a prosthetic body of this type has had to be inserted laterally into the volume of spongy bone, which has necessitated an osteotomy of the Trochanter major. The Trochanter major has then had to be secured to the prosthetic body by means of a wire loop, in order to stabilize the tensile forces in the musculature.

It has since been discovered that such a proximal resection of the proximal femur is not absolutely necessary.

It is therefore the object of the invention to provide a prosthetic body of the type described in the introductory paragraph, and a rasp instrument that can be used with it, with which a securely fixed prosthesis which is able to withstand everyday levels of stress can be achieved, with a decreased use of material in the prosthetic body, and with less invasive surgical intervention into the femur.

The invention is attained in that, in a prosthetic body of the type described in the introductory paragraph, at least the lower connection area of the support plate, to which the shaft posts are attached, is arranged horizontally, the support plate extends medially over the shaft or its cross-section, which is immediately below the area of connection, and the support plate transitions ventrally and dorsally into the shaft without overhang.

A prosthetic body of this type according to the invention is therefore supported substantially only medially over the area of the support plate which protrudes there on the cortical bone of the femur, namely especially at the point where the cortical region is particularly strong and stable, and with its greatly enlarged surface on the spongy trabeculae remaining in the residual volume of spongy bone. In contrast, the support plate has no or no significant overhang ventrally and dorsally beyond the cross-section of the shaft immediately below the connection area, so that the ventral and dorsal side areas of the support plate are arranged essentially only medially in the femur, i.e., in these areas it does not rest on the cortical bone, as was previously the case in the embodiments according to the prior art. Thus, with a prosthetic body according to the invention, a resection that is approximately 4-6 ml larger can be performed, which will not negatively affect the ingrowth of the prosthetic body, however a smaller support plate will be sufficient, as the resection is performed distally, and therefore less material can be used in the prosthetic body.

In the embodiment according to the invention, according to which the support plate transitions ventrally and dorsally into the shaft without overhang, the feature that there is no overhang is understood to mean not only a mathematically precise alignment between shaft and support plate, but also small protrusions, which are small enough that no collar forms, wherein the protrusions provide support against the cortical bone of the femur.

Support plates without overhang are accordingly understood as plates which are arranged with their ventrally and dorsally arranged lateral surfaces medially in the femur. In this case, overhangs beyond the shaft ranging from 0-10%, preferably 0-5%, referred to the thickness of the shaft can be provided is between the ventral and dorsal lateral surfaces of the support plate. Support plates with small overhangs of this type are understood as support plates which, according to the invention, are embodied without overhang, as compared with support plates as are known from the prior art.

Otherwise, a prosthetic body can be implanted into the femur in the customary method and manner known for the prosthetic bodies of this generic type, wherein the metal-free volume of the prosthetic shaft is to be filled up either with auto or artificial transplanted spongy bone.

In a particularly preferred further development, it can be provided that the prosthetic body ends laterally in the area of the support plate, or the support plate itself ends laterally in such a way that the prosthetic body or the support plate, once it has been inserted into the femur, at least predominantly does not rest against the cortical bone of the Trochanter major.

It has long been known in the art that prosthetic bodies of the type described in the introduction have a lateral and upwardly pointing projection, especially curved, laterally on the support plate, with which the known prosthetic body is supported against the cortical bone of the Trochanter major.

Through extensive research it has been found that a trochanter support of this type on the support plate can be dispensed with without this negatively affecting the stability of a prosthetic body that has grown into the bones. Thus, with a prosthetic body according to the invention, a trochanter support of this type can be dispensed with in contrast to a prosthetic body known from the prior art.

For example, for this purpose, the prosthetic body can end at the level of the support plate, or the support plate itself can end descending laterally toward the shaft. In contrast, support plates having a trochanter support ended ascending laterally, so as to be supported with this ascending area against the cortical bone of the trochanter. Thus with the embodiment according to the invention, the prosthetic body has a shape which projects less in the upper area and can therefore also be more easily implanted.

It can also be provided here that the dimensions of a prosthetic body at the level of the support plate are configured such that the prosthetic body or the support plate itself ends parallel with the longitudinal axis of a prosthesis head or its neck, which is arranged on the support plate, at a distance of 20 to 30 mm, preferably 22 to 26 mm, and particularly preferably 24 mm. These short distances ensure that laterally on the prosthetic body there is no element or area that will rest supported against the Trochanter major or its cortical bone once a prosthetic body according to the invention has been inserted.

With the prosthetic bodies according to the invention described here, it can be provided that a prosthesis head is securely arranged on the support plate. This prosthesis head has a neck area, which transitions into the upper area of the support plate, wherein a joint ball is fastened, or can be fastened, on the prosthesis head.

In an alternative and particularly preferred embodiment, it can be provided that a prosthesis head can be fastened with its neck area on a support plate. Thus a prosthesis head can also be fastened onto a prosthetic body of the invention afterward, for example, once the prosthetic body has been inserted into the femur.

In this case, it can further be provided according to the invention that a prosthesis head can be mounted in various angular positions on a support plate, for example, in at least two angular positions. These can preferably be discrete angular positions, i.e., a prosthesis head can be mounted in one or the other angular position, but not in intermediate positions.

For example, it can be provided here that the neck area or a projection or mounting piece arranged thereon can be inserted into a mounting counterpart, for example, a recess, particularly one of at least two recesses on the support plate. Such recesses, for example, two recesses, can intersect at least in their upper areas.

Such recesses can be embodied as bored holes, for example, with the bored holes extending in two or more different directions, depending upon the number of bored holes, beneath the support plate, starting from a shared opening in the support plate. A prosthesis head which is inserted with its neck area through a shared opening of this type can therefore be inserted into one of the provided bored holes, depending upon the direction of insertion, and can therefore occupy different angles in the same support plate. In this case, with a support plate of the type according to the invention as described in the introduction, which is aligned horizontally after its insertion, and is particularly arranged perpendicular to the axis of the longitudinal extension of the shaft, it is preferably provided that the neck of the prosthesis head is to be arranged at an angle of 58 degrees is relative to the prosthesis support plate, i.e., in the so-called valgus position. In this manner, overstrain between bones and implant, particularly at the Shenton line, are avoided. In another embodiment, it can also be provided to arrange the neck of the prosthesis head at an angle of 30 degrees relative to the support plate, depending upon the condition found in the patient.

It is therefore particularly preferably provided that with prosthesis heads that can be mounted, one of these two above-described angles is achieved, depending upon the direction of insertion. In this case, it is provided according to the invention that the above-described angle is achieved not only with prosthesis heads that can be mounted on the support plate, but also with prosthesis heads that are securely fastened to a support plate already during manufacture. It can therefore essentially be provided to prepare two different prosthesis heads, one with an angle of 58 degrees and one with an angle of 30 degrees between prosthesis neck and support plate. Of course, other angles are also possible, if these prove to be suitable.

In a further preferred embodiment, which can be used alone or in combination with the above-described embodiments, it is provided that the prosthesis head has at least one connecting element, particularly one connecting element in a proximal area and one connecting element in a distal area, wherein the connecting element can be embodied, for example, as a threaded bored hole, via which the prosthetic body can be connected to a vibration device. For example, such a connecting element, particularly an above-described threaded bored hole, can be arranged ventrally and/or dorsally in the support plate and/or at the lower shaft end (olive).

Coupling a vibration device to such a connecting element, particularly at two points on the prosthetic body, allows a prosthesis which has grown into a bone to be removed from the bone at a later time by applying oscillations to the prosthesis. For example, a vibration device can be used to induce vibrations in the prosthetic body, for example, via rods, which are screwed into the threaded bored holes in the dorsal and proximal regions, thereby achieving a controlled shattering of the spongy bone around the prosthetic body or its shaft. This makes it possible to draw the prosthetic body back out of the femur, once the body has been loosened in the spongy bone, for example, by applying a single, targeted blow.

To provide access to the threaded bored holes in the prosthetic body, particularly a dorsally arranged threaded bored hole at the lower end of the shaft, it is provided that access points are to be established through the bone, which will allow a vibration device to be connected.

In a further preferred embodiment, it can be provided that the neck of a prosthesis head is arranged at an angle of 4-5 degrees, particularly at an angle of 4.4 degrees, relative to the ventral end surface of the support plate. This also results in physiological advantages for a prosthesis of this type according to the invention.

In a further embodiment according to the invention, it can also be provided to insert a chip into the prosthetic body, which can be read from outside the body without contact. A chip of this type may be an RFID chip, for example. The advantages of this are that the prosthetic body that is used can be identified, for example, by a stored serial number, and that a coordinate, for example, relative to a fixed original position in the chip can be stored and can be read out from outside the body, indicating where a connection element is located relative to the original position, thereby allowing a vibration device to be fastened to the prosthetic body. Thus by reading out this coordinate information, a surgeon can identify the position in which, for example, a bored hole must be made in the bone in order to end at a connecting element, particularly a threaded bored hole, which will allow connection of a rod from the vibration device that will be used to induce the vibrations.

A rasp instrument as described in the introduction according to the invention for a prosthetic body of the above-described type according to the invention, which has rasp projections on its surface for rasping out the spongy bone at the proximal end of a femur, is characterized in that, for one, the shape of its surface matches that of the surface of the prosthetic body, at least the surface of an imaginary outer shell of the prosthetic body, which is imaginarily placed about the shaft posts. With the embodiment of a rasp instrument of this type according to the invention, it is provided that the surface of the prosthetic body is larger than that of the rasp instrument, particularly such that the cross-section of the prosthetic body is larger at every point by 0.5-4 mm, preferably by 1-3 mm, and particularly preferably by 2 mm. In this manner, during insertion of the prosthetic shaft into the spongy bone cavity, which has been generated using the smaller rasp instrument, an adequate press fit can be generated between prosthesis shaft and the inner wall areas of the cortical bone and remaining spongy bone, resulting in a stable structure and fit that can be maintained until the spongy bone has become completely incorporated into the implanted prosthetic body.

Illustrated embodiments of the invention are illustrated in the figures described in what follows. The drawings show:

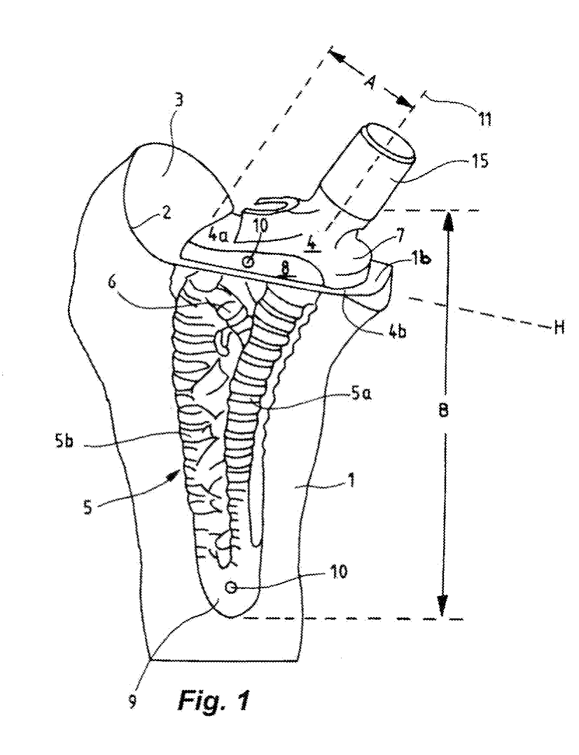

FIG. 1: a schematic diagram of a prosthetic body according to the invention following insertion into a femur;

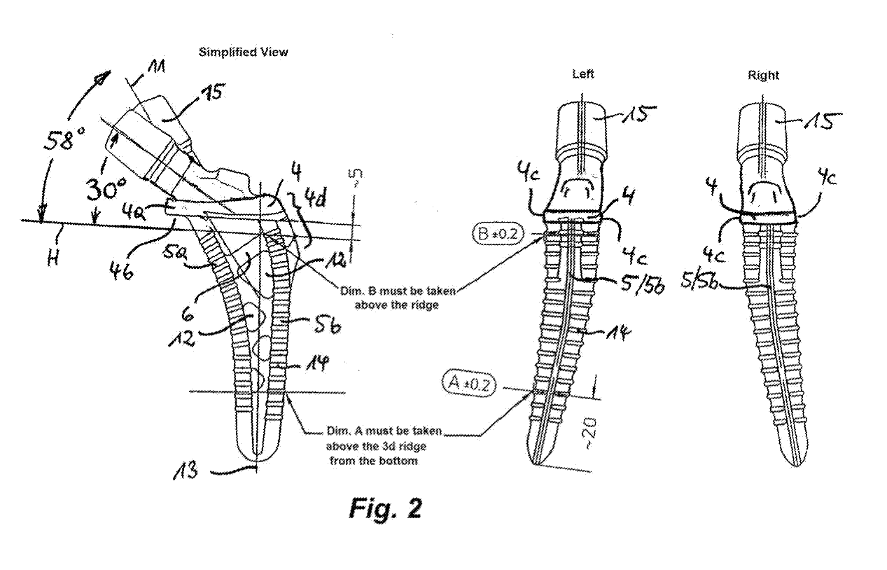

FIG. 2: several views of a prosthetic body according to the invention;

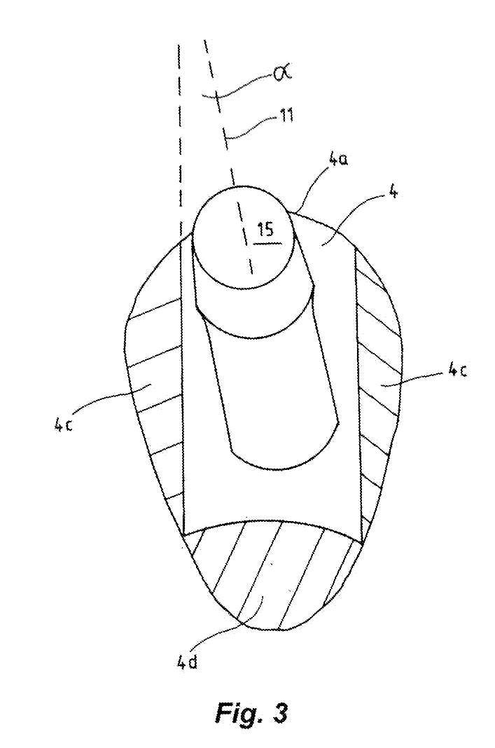

FIG. 3: the view of a prosthetic body according to the invention as compared with the prosthetic body according to the prior art.

FIG. 1 shows a prosthetic body of the type according to the invention, which is inserted into the upper proximal region of a femur 1. For this purpose, a resection of the proximal region has been performed along resection line 2, wherein a significant portion of the Trochanter major 3 has been preserved.

As is shown here, the prosthetic body has a support plate 4, below which two shaft posts 5a and 5b descend, being securely mounted on the support plate 4 at its lower connection area. For example, the upper part of the prosthetic body can be produced together with the support plate 4, and the lower part can be produced together with the shaft posts 5a and 5b, in a casting process. The two rear shaft posts are not visible due to the projection.

As is further shown here, diagonal struts 6 extend between the shaft posts 5a and 5b, allowing the shaft posts 5a and 5b to be connected to one another and stabilizing the entire shaft 5. This results in cavities between the shaft posts 5a and 5b and the struts 6, and continuous open areas 12, into which spongy bone can grow following insertion of a prosthetic body of this type. Preferably, the metal volume of the prosthetic shaft should not exceed the spongy bone volume in which the prosthetic shaft rests by more than 9-12%.

It is further shown here that, with the embodiment of this prosthetic body according to the invention, the support plate 4 has only a medial overhang or collar area 7, which extends over the medial shaft post 5a or the cross-section of the proximal end of the shaft directly below the support plate 4, particularly in such a way that the lower surface 4b of this overhanging area 7 rests on the cortical bone 1b of the femur 1, which is particularly stable at this point.

The drawing further shows that the ventral lateral surface 8 of the support plate 4, like the dorsal lateral surface of the support plate 4, which is not visible here, has no overhang relative to the shaft directly below the support plate 4, or at least has only an insignificant overhang within the context of the invention. As a result, the ventral and dorsal regions of the support plate do not rest on the cortical bone of the femur, and the support plate is instead arranged medially in the femur 1.

As is further shown, the lateral end 4a of the support plate 4 has no upwardly projecting area, such as is known from the prior art, rather the support plate 4 here essentially ends in an extension of the shaft 5 below it. Here, the support plate can have an area descending below it to the shaft 5. In particular, however, it is provided in every case according to the invention that the lateral end 4a of the support plate or the lateral region of the prosthetic body ends in such a way that, after insertion, it at least substantially does not rest against the Trochanter major 3, in other words, it is at least not substantially supported against it.

In the embodiment of the prosthetic body according to the invention shown here, it is apparent that a prosthesis head 15 is securely mounted on the support plate 4, wherein the longitudinal axis of the head forms an angle of 58 degrees from horizontal H, in which plane at least the lower side of the support plate 4 is arranged. An angle of 30 degrees could also preferably be provided here.

A joint ball which corresponds to a joint socket in the hip bone can be mounted on the prosthesis head 15 here in a known manner.

The embodiment represented here shows a further preferred development, which can be provided but need not necessarily be provided, and which consists in that, both at the distal end 9 of the prosthetic shaft 5 and at its proximal end, and in this case, particularly in the ventral lateral surface 8 of the support plate 4, a threaded bored hole 10 is arranged. Once corresponding access through the bone has been created, a rod can be screwed into a threaded bored hole 10 of this type, and can be used to introduce vibrations into the prosthetic body via a vibration device, thereby inducing vibration of the prosthetic body within the spongy bone, shattering the spongy bone around the shaft 5, so that after a prosthetic body of this type has become incorporated, it can be removed from the femur at a later time.

The embodiment according to the invention represented here further shows that the lateral end of the support plate 4 in the region 4a has a spacing parallel to the longitudinal axis 11 of the prosthetic body 15, which in this case is preferably chosen as A=20-30 mm, more preferably 22-26 mm, and particularly preferably 24 mm. This also ensures that, once the prosthetic body has been inserted, the prosthetic body and/or its support plate 4 essentially do not rest against the Trochanter major.

The measurement B between the distal end of the prosthetic body and an instrument attachment can in this case preferably be chosen as B=104 mm. Other lengths are also possible.

FIG. 2 shows several views of prosthetic bodies according to the invention, in which it is apparent that the support plate 4 has a projection beyond the shaft, in this case particularly the medial shaft post 5a, only in its medial area 4a, which faces the hip bone, so that the underside 4b of this medial collar 4a is able to rest on the cortical bone of the femur. The diagrams labeled “left” and “right” on the right side of the drawing, showing the prosthetic body according to the invention for a left and a right femur, show, in a lateral view, that the ventral and dorsal lateral surfaces 4c either have no overhang, or overhang only insubstantially in a range of less than 10% in relation to the shaft 5 directly below the support plate 4.

The representation chosen here also shows that diagonal struts 6 extend between the medial shaft post 5a and the lateral shaft post 5b, and that cavities or free open areas 12 are created in the intermediate areas, into which spongy bone material can grow.

Also apparent in FIG. 2 is a recess with which a prosthesis head 15 can be inserted into the support plate 4 in various angular positions. In this drawing, two angular positions are shown, superimposed, in which the prosthesis head 15 is arranged either at an angle of 30 degrees or at an angle of 58 degrees from horizontal H, i.e., the alignment at least of the underside of the support plate 4. The support plate 4 is in turn aligned with its underside perpendicular to the longitudinal axis 13 of the shaft 5, shown vertically here.

FIG. 2 also clearly shows, particularly in the diagram on the left side, that the support plate 4 is embodied as descending laterally, i.e., above the shaft post 5b, in this case particularly in the area 4d. This sloped lateral surface 4d shown here has a parallel spacing particularly to the center axis 11 of the prosthesis head 15, at an angle of 58 degrees, with the spacing preferably lying in the above-described range of 20-30 mm, preferably 22-26 mm and particularly preferably 24 mm.

It is also important to note here that the support plate 4 has no trochanter support area projecting upward laterally, and also has no trochanter support area extending laterally beyond the extension of the shaft post 5b.

As in FIG. 1, the FIG. 2 also show that along the axial extension of the shaft posts 5a and 5b, respectively spaced from one another, thickened areas, so-called anchoring bands 14, are provided, which also serve to increase stability following growth into the femur. The most important function of these anchoring bands is surface enlargement.

FIG. 3 shows a direct comparison from a plan view of a prosthetic body according to the invention with a prosthetic body as is known from the prior art. The surface areas 4c, 4d shown with hatch marks here indicate the outer dimensions of a support plate 4 for a prosthetic body according to the prior art, with a highly proximal resection. The resection extends horizontally, but lies in the proximal area of the femur neck, and partially in the distal area of the femur head.

As is shown here, a prosthetic body according to the invention differs from the known prosthetic body in that ventral and dorsal lateral areas 4c are eliminated from the known support plate 4 to create ventral and dorsal lateral surfaces of the support plate 4 which do not extend, or at least extend only insignificantly within the context of the invention, beyond the shaft below them. A lateral area 4d is also dispensed with, so that the support plate 4 extends beyond the shaft below it only in the medial area 4a. In a view from the top, in contrast, the ventral, dorsal and lateral boundaries of the support plate are either flush with the shaft below them, particularly with respect to the ventral and dorsal areas, or in any case extend insignificantly, or are even set back, particularly with respect to the lateral area.

FIG. 3 also shows that it is provided according to the invention, in one embodiment, that the longitudinal axis 11 of the prosthesis head 15 forms an angle a with the ventral lateral surface of the support plate 4, which angle preferably ranges in measurement from 4-5 degrees, and can particularly preferably be chosen as 4.4 degrees.

With respect to all embodiments, it is to be noted that those technical features specified in connection with one embodiment can be used, or are used, not only in that specific embodiment, but also in the respective other embodiments. All disclosed technical features of this description of the invention are considered to be essential to the invention and may be used in any combination, or separately. In addition, throughout the disclosure, when it is mentioned that a feature can be provided or a process step can be performed, an embodiment of the invention is also to be understood in which the relevant feature is provided or a relevant process step is performed.

1. A prosthetic body for an upper leg prosthesis comprising a shaft having a proximal support plate for a prosthesis head, wherein at least two proximally spaced and distally converging shaft posts are mounted on the support plate, descending below the same, and are connected by diagonal struts, and wherein cavities or free open areas are formed between the shaft posts and the struts, into which spongy bone can grow, wherein
at least the lower connection area of the support plate, on which the shaft posts are mounted, is arranged horizontally,
the support plate extends medially beyond the cross-section of the shaft, which is immediately below the area of connection, and
the support plate transitions ventrally and dorsally into the shaft without an overhang.

2. The prosthetic body according to claim 1, wherein the prosthetic body ends laterally in the area of the support plate such that when the prosthetic body has been inserted into the femur, at least predominantly does not rest against the cortical bone of the Trochanter major.

3. The prosthetic body according to claim 1, wherein the prosthetic body ends at the level of the support plate and the support plate ends descending laterally toward the shaft.

4. The prosthetic body according to claim 1, wherein the prosthetic body ends at the level of the support plate and the support plate ends parallel to the longitudinal axis of the prosthesis head or its neck at a spacing of 20 to 30 mm.

5. The prosthetic body according to claim 1, wherein a prosthesis head with its neck area can be mounted on the support plate to- or on a mounting area arranged thereon, particularly in at least two different angular positions.

6. The prosthetic body according to claim 5, wherein the neck area or a projection arranged on the area can be inserted into a recess, particularly one of at least two recesses, on the support plate.

7. The prosthetic body according to claim 6, wherein two recesses intersect at least partially, at least in their upper regions, particularly wherein each recess is embodied as a bored hole, and the bored holes extend in two different directions starting from a shared opening in the support plate.

8. The prosthetic body according to claim 1, wherein it has at least one connecting element, particularly one connecting element in the proximal area and one in the distal area, particularly a threaded bored hole, with which the prosthetic body can be connected to a vibration device.

9. The prosthetic body according to claim 8, wherein a connecting element is arranged ventrally or dorsally in the support plate or at the lower shaft end.

10. The prosthetic body according to claim 1, wherein the neck of a prosthesis head is arranged at an angle of 4-5 degrees relative to the ventral end surface of the support plate.

11. The prosthetic body according that claim 1, wherein an RFID chip for storing and reading out an identification number or coordinate information for a connecting element, is arranged on or in the body.

12. A rasp instrument for a prosthetic body according to claim 1, the instrument having a surface which has rasp projections, and which has the same shape as the surface of the prosthetic body, wherein the surface of the prosthetic body is larger than that of the rasp instrument, particularly such that the cross-section of the prosthetic body is larger at every point by 0.5 to 4 mm.

13. A vibration device for generating mechanical vibrations, wherein it has at least one rod that can be connected by connecting elements to a prosthetic body, particularly according to claim 8, in order to introduce generated mechanical vibrations into the prosthetic body.
